(19) United States
(12) Patent Application Publication
(10) Pub. No.: US 2002/0183556 A1
(43) Pub. Date: Dec. 5, 2002

(54) Ruthenium catalyst for the hydrogenation of diaminodiphenylmethane to diaminodicyclohexylmethane

(76) Inventors: Andreas Schulze Tilling, League City, TX (US); Thomas Prinz, Leverkusen (DE); Jurgen Kintrup, Leverkusen (DE)

Correspondence Address: BAYER CORPORATION, PATENT DEPARTMENT, 100 BAYER ROAD, PITTSBURGH, PA, 15205, US

(21) Appl. No.: 10/122,834

(22) Filed: Apr. 15, 2002

(30) Foreign Application Priority Data
Apr. 19, 2001 (DE) 10119136.7

(51) Int. Cl. B01J 23/56; C 07C 2 9/72

(52) U.S. Cl. 564/451; 502/332

(57) ABSTRACT

The invention relates to ruthenium catalysts for the hydrogenation of diaminodiphenylmethane to diaminodicyclohexylmethane, particularly with a proportion of trans,trans-4,4′-diaminodicyclohexylmethane of from 17 to 24%, in a continuously operated suspension reactor, where ruthenium is applied to a support of high-purity aluminum oxide that preferably has a sodium content of less than 0.05% by weight, a particle size of from 5 to 150 μm, and a BET specific surface area of from 30 to 300 m²/g.

### BACKGROUND OF THE INVENTION

The present invention relates to ruthenium catalysts for the hydrogenation of diaminodiphenylmethane (MDA) to diaminodicyclohexylmethane (PACM) in a continuously operated suspension reactor, where ruthenium is applied to a support of high-purity aluminum oxide.

PACM is prepared industrially by hydrogenating MDA. PACM is used, for example, for the preparation of surface coatings, primarily as a precursor for the surface-coating raw material diisocyanatodicyclohexyl-methane. The isomer ratio is of particular importance for a number of applications.

EP 639,403 A2 discloses a catalyst for the preparation of PACM with a low proportion of trans,trans isomer by hydrogenating MDA. This catalyst has a thin ruthenium- or rhodium-containing layer on a special support, namely a calcined or superficially rehydrated transition alumina, particularly hydrargillite or bayerite.

EP 639,403 A2 describes the deactivation of the catalyst by higher molecular weight constituents of the reaction mixture and the adjustment of a low proportion of trans,trans isomer in the product as a problem in the industrial preparation of PACM. The use of the special catalyst is intended to solve these problems. However, the special catalyst is primarily suitable for use in reactors with a fixed catalyst bed in which the catalyst cannot be exchanged during operation. In addition, a large part of the reactor volume is occupied by the inactive core of the coated catalyst used and is no longer available as reaction volume.

Hydrogenations in discontinuously operated suspension reactors have already been described. Suspension reactors have the advantage that the spent catalyst can be readily exchanged.

It was therefore an object of the invention to provide a catalyst which permits the hydrogenation of MDA to PACM, particularly with a low proportion of trans,trans4,4′-diaminodicyclohexylmethane, in a continuously operated suspension reactor with a high space-time yield and a high catalyst service life.

Surprisingly, it has been found that by using high-purity aluminum oxide as support, it is possible to prepare pulverulent, suspendable ruthenium catalysts with which MDA can be hydrogenated with a high space-time yield to give PACM with a low proportion of trans,trans-4,4′-diaminodicyclohexylmethane with a high catalyst service life.

### SUMMARY OF THE INVENTION

The invention provides ruthenium catalysts for the hydrogenation of diaminodiphenylmethane (MDA) to diaminodicyclohexylmethane (PACM) in a continuously operated suspension reactor, wherein the catalyst is ruthenium applied to a support of high-purity aluminum oxide.

### DETAILED DESCRIPTION OF THE INVENTION

The aluminum oxide used as support material is particularly characterized by comprising only small amounts of alkali metals, in particular sodium.

The fact that catalysts on a high-purity support, particularly with a particularly low sodium content, are advantageous as hydrogenation catalysts is surprising since in other cases it is these very alkali metal compounds that are added as promoters. For example, WO 9608462 μl describes a process for the catalytic hydrogenation of aromatic amines in the presence of a noble metal catalyst and lithium hydroxide as promoter.

Preferably, the high-purity aluminum oxide which is used according to the invention a support material has a sodium content of less than 0.05% by weight (particularly preferably of at most 0.02% by weight), calculated as Na₂O.

Such high-purity aluminum oxides are prepared industrially, for example, by the hydrolysis of aluminum alkoxides. Aluminum oxides that have hitherto customarily been used as support materials for hydrogenation catalysts have a Na₂O content in the range from 0.1 to 0.5% by weight.

The special supports for hydrogenation catalysts known from EP 639,403 A2, namely calcined or superficially rehydrated transition aluminas (particularly hydrargillite or bayerite) are likewise based on aluminum oxide. However, according to EP 639,403 A2, only those supports that have a certain basic buffer capacity are advantageous for ruthenium-containing hydrogenation catalysts. The buffer capacity of the aluminum oxides according to the invention does not lie within the range regarded as advantageous. The advantages of an aluminum oxide support with a low sodium content is not discussed in EP 639,403 A2. In addition, it is demonstrated in EP 639,403 A2, particularly for fixed-bed catalysts, that a shell-like distribution of the ruthenium on the catalyst support is advantageous. In contrast, for the catalysts according to the invention, particularly when they are used as powders in a suspension reactor, it is particularly advantageous if the ruthenium is distributed over the entire cross section of the support particles. This leads to better activity and service life of the catalyst.

In the case of the ruthenium catalysts according to the invention, the ruthenium is therefore preferably distributed over the entire cross section of the support particles.

The high-purity aluminum oxide used as support material is preferably a powder with an average particle diameter of from 5 to 150 μm, particularly preferably from 10 to 120 μm, especially preferably from 30 to 100μm.

The high-purity aluminum oxide preferably has a BET specific surface area of from 30 to 300 m²/g, particularly preferably from 70 to 200 m²/g, especially preferably from 100 to 160 m²/g.

The pore volume of the high-purity aluminum oxide is, taking into consideration pores with a diameter of <10,000 nm, preferably 0.1 to 1.5 ml/g, particularly preferably 0.3 to 0.7 ml/g.

In addition to high-purity aluminum oxide, other support materials that can be used are aluminum-containing, low-alkali metal mixed oxides that have the same physical properties as high-purity aluminum oxide.

The ruthenium catalysts according to the invention are preferably in powder form.

The ruthenium content is preferably 1 to 10% by weight, particularly preferably 4 to 8% by weight.

As well as comprising ruthenium, the ruthenium catalysts according to the invention can also comprise other metals, for example, rhodium.

The ruthenium catalysts are preferably characterized by good filterability and by the fact that the catalyst can, following its use and removal of the product solution, be reused for the hydrogenation of diaminodiphenylmethane (MDA) to diaminodicyclohexylmethane (PACM) in a continuously operated suspension reactor.

Preference is given to ruthenium catalysts that are suitable as catalysts for the hydrogenation of diaminodiphenylmethane (MDA) that comprise, in addition to MDA, higher molecular weight aromatic amines, to diaminodicyclohexylmethane (PACM).

The catalysts according to the invention can be prepared, for example, by suspending the high-purity aluminum oxide support according to the invention in water and then adding an aqueous solution of a ruthenium compound or a ruthenium salt, such as, for example, ruthenium chloride or ruthenium nitrosyl nitrate. The ruthenium is left to adsorb onto the support, and then a base (e.g., sodium carbonate, sodium hydroxide solution, or lithium hydroxide) is added to precipitate the ruthenium. A reducing agent (e.g., formaldehyde, sodium formate, or hydrazine) can be added. The mixture is then filtered, and the catalyst is washed until free from chloride and sodium and dried. The dried pulverulent catalyst can additionally be reduced with hydrogen at temperatures of from 100 to 250° C. in a reducing furnace and passivated with inert gas/air mixture. The catalyst can, however, also be suspended in a solvent in a hydrogenation reactor in which the hydrogenation of MDA to PACM is to take place and reduced there with hydrogen.

The ruthenium catalysts according to the invention are used for the hydrogenation of diaminodiphenylmethane (MDA) to diaminodicyclohexylmethane (PACM) in a continuously operated suspension reactor.

The ruthenium catalysts are preferably used for the preparation of diaminodicyclohexylmethane (PACM) with a proportion of trans,trans-4,4′-diaminodicyclohexylmethane of from 17 to 24%.

The use of the ruthenium catalysts according to the invention for the hydrogenation of diaminodiphenylmethane (MDA) to diaminodicyclohexylmethane (PACM) in a continuously operated suspension reactor takes place, for example, at a hydrogen pressure of from 50 to 400 bar, preferably from 100 to 200 bar.

Hydrogen is advantageously added in an excess of from 5 to 200%, preferably from 20 to 100% of theory.

The temperature is, for example, from 130 to 190° C., preferably from 150 to 180° C.

The catalyst according to the invention can, for example, be used in an amount of from 1 to 10% by weight, preferably 3 to 8% by weight, based on the reaction mixture.

The parameters catalyst concentration, temperature, and residence time in the reactor can be used to adjust the content of trans,trans isomer in the product. In this way, products with a low proportion of trans,trans isomer, particularly with a proportion between 17 and 24%, can be achieved. For example, at a given temperature and catalyst concentration, the proportion of trans,trans isomer in the product can be adjusted by adapting the residence time of the reaction mixture in the reactor.

The hydrogenation is carried out in a suspension reactor, preferably a stirred-tank reactor or a bubble column, particularly preferably in a cascade of two or more serially connected stirred-tank reactors or bubble columns.

The mixing of MDA starting material with the catalyst used and the hydrogen required for the hydrogenation is carried out when using stirred-tank reactors as suspension reactors by means of a stirrer and when using bubble columns as suspension reactors by introducing hydrogen at high speed and generating a turbulent flow within the reactor.

The hydrogenation can be carried out with or without the addition of organic solvents. Suitable solvents are, for example, alcohols, preferably secondary alcohols (e.g., isobutanol, cyclohexanol, or methylcyclohexanol) or tertiary alcohols (e.g., tert-butanol), particularly preferably tertiary alcohols.

The procedure preferably involves conveying the catalyst through the suspension reactor together with the reaction mixture. The product mixture is then cooled, excess hydrogen is eliminated, and the catalyst is filtered. Following removal of the catalyst, the optionally used solvent can be separated from the product by distillation and returned to the hydrogenation process.

Catalysts according to the invention are characterized, even after prolonged use, by good filterability and high mechanical stability.

Following removal of the product solution, the catalyst is preferably reused for the hydrogenation of MDA.

With regard to catalyst activity and service life, it is advantageous to wash the catalyst with an inert solvent after the product solution has been separated off. This enables the catalyst surface to be freed from deposits of higher molecular weight reaction products.

If the catalyst activity decreases after a relatively long period of operation, some of the catalyst can be removed from the system and be replaced by fresh catalyst, meaning that a plant for carrying out the process according to the invention can be operated with constant average catalyst activity and constant throughput.

The invention is illustrated in more detail below by reference to examples. The examples represent individual embodiments of the invention, but the invention is not limited to the examples. Those skilled in the art will readily understand that known variations of the conditions of the following procedures can be used. Unless otherwise noted, all temperatures are degrees Celsius and all percentages are percentages by weight.

### EXAMPLES

### Example 1

### Catalyst Preparation

855 g of aluminum oxide (Al₂O₃) with a BET specific surface area of 160 m²/g, an average particle diameter of 50 μm, and a sodium content of 0.02% by weight, calculated as Na₂O, were suspended in 3,600 ml of demineralized water with stirring in a 10 liter reaction vessel. The mixture was then stirred for 10 minutes. 360 ml of a solution consisting of 225 g of ruthenium chloride solution with a 20% by weight ruthenium content (corresponding to 45 g of ruthenium) and demineralized water were added to the suspension with stirring. The mixture was then after-stirred for 1 hour. About 695 g of a 10% strength by weight sodium hydroxide solution was pumped into the suspension with stirring over the course of 40 minutes until a pH of 8 was established. The mixture was then after-stirred for 1 hour and 10% strength by weight sodium hydroxide solution was again used to adjust the pH to 8. A solution of 200 g of hydrazine hydrate in 4 liter of water was then added, and the mixture was stirred for a further hour. The resulting catalyst suspension was filtered and washed with demineralized water until the water which ran off was neutral and chloride-free. The filter cake was sucked dry for 30 minutes. Finally, the catalyst was dried in a vacuum drying cabinet at 110° C.

### EXAMPLE 2

### Catalyst Test in a Continuously Operated Stirred-Tank Reactor

MDA was hydrogenated in a continuously operated stirred-tank reactor having a reaction volume of 330 ml. A pulverulent catalyst prepared according to Example 1 was introduced into the stirred-tank reactor in a catalyst concentration of 5% by weight. MDA was used in technical-grade quality (so-called MDA 90/10) with a proportion of about 10% of higher molecular weight components as 33% strength by weight solution in isobutanol. The MDA 90/10-isobutanol mixture was metered into the reactor from a storage container. The reactor pressure was kept constant at 150 bar by continuously replenishing hydrogen. In the experiment, a temperature of 150° C. was set.

The overflow of the reaction mixture passed into a further container, from which samples were taken for analysis. By varying the delivery capacity of the dosing pump, a variety of average residence times were established. The samples were analyzed using gas chromatography.

The contents of MDA, [H₆]-MDA (i.e., 4-aminocyclohexyl-4-amino-phenylmethane), and PACM and the proportion of trans,trans-PACM (tt proportion) are given in Table 1.

TABLE 1

| | |
|---|---|
| Temperature [° C.] | 150 |
| Residence time [min] | 61 |
| Throughput [g of PACM per l and h] | 247 |
| PACM [%] | 88 |
| tt proportion [%] | 24 |
| [H₆]-MDA [%] | 1.5 |
| MDA [%] | 0.1 |
| Higher molecular weight components [%] | 10 |

### Example 3

### Catalyst Test in a Continuously Operated Cascade of 3 Stirred-Tank Reactors

Example 2 was repeated, although the residence time was shortened so that conversion was only partial. The product was then passed through the reactor a further two times. The product corresponded to the product obtained in a cascade of three stirred-tank reactors.

The contents of MDA, [H₆]-MDA, and PACM and the proportion of trans,trans-PACM (tt proportion) are given in Table 2.

TABLE 2

| | |
|---|---|
| Temperature [° C.] | 150 |
| Residence time [min] | 45 |
| Throughput [g of PACM per l and h] | 330 |
| PACM [%] | 89 |
| tt proportion [%] | 19 |
| [H₆]-MDA [%] | 0.9 |
| MDA [%] | 0.1 |
| Higher molecular weight components [%] | 10 |

The experiment shows that if a cascade of three reactors is used, complete conversion and a trans,trans proportion in the region of 20% can be achieved at the same time, and also a high space-time yield is achieved.

### Example 4

### Catalyst Test in a Discontinuously Operated Stirred-Tank Reactor

MDA was hydrogenated in a discontinuously operated stirred-tank reactor having a reaction volume of 330 ml. A pulverulent catalyst prepared according to Example 1 was introduced into the stirred-tank reactor in a catalyst concentration of 5% by weight. MDA was used in technical-grade quality (so-called MDA 90/10) with a proportion of about 10% of higher molecular weight components as 33% strength by weight solution in isobutanol. 330 ml of the MDA 90/10-isobutanol mixture were metered into the reactor from a storage container. The reactor pressure was kept constant at 150 bar by continuously replenishing hydrogen. In the experiment, a temperature of 150° C. was set.

After various residence times of the reaction mixture in the reactor, samples were taken from the reaction mixture for analysis. The samples taken were analyzed by means of gas chromatography.

The contents of MDA, [H₆]-MDA, and PACM and the proportion of trans,trans-PACM (tt proportion) are given in Table 3.

TABLE 3

| | | | |
|---|---|---|---|
| Temperature [° C.] | 150 | 150 | 150 |
| Residence time [min] | 51 | 111 | 171 |
| Throughput [g of PACM per l and h] | 282 | 131 | 81 |
| PACM [%] | 84 | 85 | 81 |
| tt proportion [%] | 14 | 24 | 37 |
| [H₆]-MDA [%] | 3.1 | 0.2 | 0.3 |
| MDA [%] | 0.5 | 0.1 | 0 |
| Higher molecular weight components [%] | 12 | 14 | 18 |

What is claimed is:

1. A ruthenium catalyst for the hydrogenation of diamino-diphenylmethane to diaminodicyclohexylmethane in a continuously operated suspension reactor, wherein the catalyst is ruthenium applied to a support of high-purity aluminum oxide.

2. A ruthenium catalyst according to claim 1 wherein the high-purity aluminum oxide has a sodium content of less than 0.05% by weight, calculated as Na₂O.

3. A ruthenium catalyst according to claim 1 wherein the high-purity aluminum oxide is a powder having an average particle diameter of from 5 to 150 μm.

4. A ruthenium catalyst according to claim 2 wherein the high-purity aluminum oxide is a powder having an average particle diameter of from 5 to 150 μm.

5. A ruthenium catalyst according to claim 1 wherein the high-purity aluminum oxide is a powder having a BET specific surface area of from 30 to 300 m²/g.

6. A ruthenium catalyst according to claim 2 wherein the high-purity aluminum oxide is a powder having a BET specific surface area of from 30 to 300 m²/g.

7. A ruthenium catalyst according to claim 4 wherein the high-purity aluminum oxide is a powder having a BET specific surface area of from 30 to 300 m²/g.

8. A ruthenium catalyst according to claim 1 that it is a powder.

9. A ruthenium catalyst according to claim 1 wherein the ruthenium content is 1 to 10% by weight.

10. A ruthenium catalyst according to claim 1 wherein the ruthenium is distributed over the entire cross section of the support.

11. A ruthenium catalyst according to claim 10 wherein support is in particle form.

12. A ruthenium catalyst according to claim 1 that can be readily filtered.

13. A process comprising hydrogenating diaminodiphenyl-methane to diaminodicyclohexylmethane in a continuously operated suspension reactor in the presence of a ruthenium catalyst according to claim 1.

14. A process according to claim 13 wherein the diaminodicyclohexylmethane has a proportion of trans,trans4,4′-diaminodicyclohexylmethane of from 17 to 24%.

15. A process according to claim 13 wherein the diaminodicyclohexylmethane additionally comprises higher molecular weight aromatic amines.

16. A process comprising
(1) hydrogenating diaminodiphenylmethane to diaminodicyclohexylmethane in a continuously operated suspension reactor in the presence of a ruthenium catalyst according to claim 1,
(2) separating the diaminodicyclohexylmethane from the catalyst, and
(3) reusing the catalyst for the hydrogenation of diaminodiphenylmethane to diaminodicyclohexylmethane in a continuously operated suspension reactor.
